(19) United States
(12) Patent Application Publication
(10) Pub. No.: US 2010/0092416 A1
(43) Pub. Date: Apr. 15, 2010

(54) METHOD FOR THE COSMETIC TREATMENT OF KERATIN MATERIALS

(76) Inventors: Gustavo Luengo, Montevrain (FR); Gwenaëlle Jegou, Saint Michel Sur Orge (FR); Philippe Barbarat, Bois-colombes (FR); Michel Philippe, Wissous (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/527,258

(22) PCT Filed: Feb. 18, 2008
(86) PCT No.: PCT/FR08/50264

§ 371 Date: Dec. 23, 2009

Related U.S. Application Data
(60) Provisional application No. 60/904,177, filed on Mar. 1, 2007.

(30) Foreign Application Priority Data
Feb. 16, 2007 (FR) 0753314

(51) Int. Cl. A61K 8/64; A61K 8/72; A61Q 5/06

(52) U.S. Cl. 424/70.6; 424/70.14; 510/119

(57) ABSTRACT

The present invention relates to a cosmetic treatment method for keratin materials, especially the hair, comprising the application of a cosmetic composition which comprises a polymer having a polymer backbone comprising amino acid monomer units with cationic function, and comprising hydrophilic grafts bonded covalently to said backbone, over all or part of said backbone, said hydrophilic grafts comprising the repetition of at least two hydrophilic monomer units of formula —CH₂—CH₂—O—.

The present invention relates to a method of cosmetic treatment, especially hair treatment, of keratin materials, especially the hair, employing hair management compositions comprising graft polyamino acid polymers.

It is well known that the hair is sensitized, in other words damaged and/or weakened, to various degrees by the action of atmospheric agents, especially light, and also by the repeated action of various mechanical or chemical treatments, such as permanent wave treatments, hair straightening, colouring and bleaching. These aggressive treatments alter the hair fibre and so may attenuate its mechanical properties such as the tensile strength, breaking load and elasticity. More particularly, instances of degradation of the fibre have been observed after these treatments: the hair becomes more hydrophilic and may lose part of the scales, a loss which is manifested in great difficulty in disentangling and styling the hair, and also a loss of softness.

To combat the mechanical degradation of the keratin of the hair by light, proposals have already been made to use certain substances which are capable of filtering luminous radiation, such as 2-hydroxy-4-methoxybenzophenone-5-sulphonic acid or its salts (FR-A-2 627 085) or lactoferrin (FR-A-2 673 839).

In order to preserve and/or reinforce the mechanical properties of the hair, and especially its tensile strength and its elasticity, proposals have also been made to use flavonoids, especially in patent application FR-A-2719467.

The applicant has now discovered, unexpectedly and surprisingly, that certain graft polyamino acid polymers may allow protection and repair of keratin materials, especially damaged keratin materials, an activity which is manifested, in the case of hair, in an improvement in the disentangling properties, on dry and wet hair, and an improvement in the touch (softness).

The present invention accordingly provides a hair treatment method for the hair, comprising the application of a cosmetic composition comprising, in a cosmetically acceptable medium, at least one polymer comprising a polymer backbone comprising amino acid monomer units with cationic function, and comprising hydrophilic grafts bonded covalently to said backbone, over all or part of said backbone, said hydrophilic grafts comprising the repetition of at least two hydrophilic monomer units of formula —CH₂—CH₂—O—.

Thus it has been demonstrated that polymers whose architecture includes a polymer backbone comprising polymerized amino acid units, obtained from the polymerization of at least one, or two or more, amino acids, with hydrophilic grafts grafted to said backbone, make it possible, following application to keratin materials, to obtain the desired properties; more particularly, these polymers can advantageously be applied to the hair, and more particularly to sensitized hair, especially damaged or weakened hair.

The cosmetic treatment of keratin materials with the composition according to the invention therefore makes it possible to provide repair properties, which may be manifested in improved disentangling and in softness to the touch, in the case of hair.

It has in fact been observed that this class of polymers improves the quality of the keratin fibres, more particularly their disentangling, irrespective of whether the hair is still wet or has already been dried.

It has also been observed that the compositions according to the invention are able to impart gloss to keratin materials, especially to the hair.

The compositions according to the invention may also make it possible to reinforce the keratin materials, and especially to improve their resistance to tensile mechanical forces imposed on them, in the course of combing, for example, more particularly on weakened or sensitized hair. An illustrative term for this is anti-breakage effect.

In the present invention the term “keratin material” encompasses especially the skin, lips, nails, head hair and body hair, eyelashes and eyebrows.

The polymers falling within the scope of the invention are known in the literature. For instance there are known polymers based on poly(amino acids) with hydrophilic grafts, such as polylysine with polyethylene oxide (or PEG) grafts, which are used, for example, in the biomedical sector to reduce immunological recognition between cells/tissues, as described in U.S. Pat. No. 5,462,990; or else for improving analytical recognition, especially the selectivity and sensitivity of diagnostic devices, as described in WO00/65352.

Also known, from US2002/0016304, are poly(amino acid) polymers with hydrophilic grafts, especially polylysine with dextran grafts, for stabilizing nucleic acids such as DNA.

The polymers according to the invention therefore possess a poly(cationic amino acid) polymer backbone, in other words a backbone comprising principally a repetition of cationic amino acid monomer units.

The polymer backbone may be linear, branched, especially star-branched or hyperbranched, dendrimeric, or else crosslinked; preferably, it is linear.

By cationic amino acids are meant any compound comprising an amino acid unit and additionally at least one cationic function, in other words a primary, secondary, tertiary or quaternary amine which can be protonated at a pH of between 3 and 12. The polymer backbone thus comprises at least one function capable of possessing a cationic charge within a pH range between 3 and 12.

Examples of a cationic function include the following:

guanidino and amidino moieties, of formula:

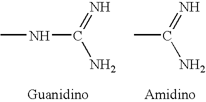

groups of formula —NRR′ or —N⁺RR′R″, where R, R′ and R″ represent, independently of one another, alternatively (i) a hydrogen atom, or (ii) an optionally aromatic, saturated or unsaturated, linear, branched or cyclic alkyl group containing 1 to 18 carbon atoms and able to contain 1 to 10 heteroatoms selected from O, N, S, F, Si and P, or (iii) R and R′ form, with the nitrogen atom, a first, optionally aromatic, saturated or unsaturated ring containing in total 5, 6, 7 or 8 atoms, and especially 4, 5 or 6 carbon atoms and/or 2 to 4 heteroatoms selected from O, S and N, it being possible for said first ring to be fused with one or more other, optionally aromatic, saturated or unsaturated rings each containing 5, 6 or 7 atoms, and especially 4, 5, 6 or 7 carbon atoms and/or 2 to 4 heteroatoms selected from O, S and N;

the groups:

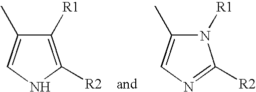

in which R1 and R2 may be, independently of one another, selected from hydrogen; an optionally aromatic, saturated or unsaturated, linear, branched or cyclic alkyl radical containing 1 to 18 carbon atoms and able to contain 1 to 10 heteroatoms selected from O, N, S, F, Si and P; or else it being possible for R1 and R2 together to form an optionally aromatic, saturated or unsaturated ring containing in total 5, 6, 7 or 8 atoms, especially 5 or 6 carbon atoms and/or 2 to 4 heteroatoms selected from O, S and N.

The cationic function may especially be selected from the groups NH₂, guanidino, and also

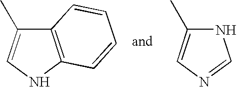

The cationic amino acid monomers are preferably selected from lysine, ornithine, arginine, histidine, glutamine and tryptophan, and a mixture thereof.

The polymer backbone according to the invention may be a homopolymer, more particularly of polylysine type, especially poly(ε-lysine), poly(α-lysine), poly(L-lysine), poly(D-lysine), poly(D,L-lysine); or of polyornithine type, especially poly(L-ornithine), poly(D-ornithine), poly(D,L-ornithine); or of polyarginine type, especially poly(L-arginine), poly(D-arginine), poly(D,L-arginine); or of polyhistidine type, especially poly(L-histidine), poly(D-histidine), poly(D,L-histidine); or of polyglutamine type, especially poly(L-glutamine), poly(D-glutamine), poly(D,L-glutamine); or of polytryptophan type, especially poly(L-tryptophan), poly(D-tryptophan), poly(D,L-tryptophan).

The polymer backbone may also be a copolymer, which may be composed solely of cationic amino acid monomers, or of cationic amino acid monomers in a mixture with additional monomers.

Accordingly the polymer backbone may be a copolymer prepared from a mixture of cationic amino acids, for example of poly(lysine-co-ornithine) type.

It may also comprise non-cationic amino acid monomers, such as alanine, especially the D and L isomers; leucine or isoleucine, especially L-leucine, L-isoleucine, D-isoleucine; serine, especially the D and L isomers; threonine, especially L-threonine; cysteine, especially the D and L isomers; aspartic acid, especially the D and L isomers; glycine, especially the D and L isomers; valine, especially the D and L isomers; asparagine, especially the D and L isomers; phenylalanine, especially the D and L isomers; tyrosine, especially the D and L isomers; glutamic acid, especially the D and L isomers; proline, especially the D and L isomers.

The polymer backbone may more particularly be poly(ornithine-co-serine), poly(lysine-co-tyrosine), poly(lysine-co-alanine) and poly(leucine-co-lysine).

When it is composed only of cationic or non-cationic amino acid monomers, the polymer backbone may be a random, alternating or sequenced (block) copolymer.

When it comprises other types of monomers, such as those mentioned above, the polymer backbone is preferably a block copolymer.

The polymer backbone may therefore comprise additional monomers or polymers of polyalkylene glycol type, and especially polyethylene glycol (PEG).

Mention may therefore be made of poly(lysine)-b-PEG block copolymers, poly(ornithine)-b-PEG block copolymers and poly(ornithine-co-serine)-b-PEG block copolymers. These polymers may be synthesized by known polymerization methods. Thus, for example, the polymerization of anhydrides of carbobenzoxylysine-N-carboxylic acid and of benzylserine-N-carboxylic acid in the presence of a PEG bearing a terminal NH₂ function (MW 200 to 25,000 g/mol) results in a poly(lysine-co-serine)-b-PEG block copolymer.

The polymer backbone may also comprise vinyl monomers, especially (meth)acrylic and/or (meth)acrylamide monomers, and/or saccharide, ester, ether, carbonate, urethane or urea monomers, and a mixture thereof.

In this case the polymer backbone may comprise one or more poly(amino acid) blocks, for example polylysine blocks, and one or more polyvinyl, polysaccharide, polyester, polyether, polyurethane and/or polyurea blocks.

Mention may be made more particularly as polymer backbone of polylysine-b-polyester or polylysine-b-polyurethane copolymers.

The polymer backbone may also be of poly(lactic acid-co-lysine) or poly(lysine-co-poly(N-hydroxypropylaminoethyl-D,L-aspartamide)) type.

These polymers forming the polymer backbone may be prepared by any means known to a person skilled in the art.

When the backbone contains only cationic or non-cationic amino acids, a general polymerization method, especially, comprises the initiation, via an initiator, of NCA (N-carboxyanhydride) alpha-amino acid monomers, especially NCA L-lysine, NCA L-ornithine, NCA glutamic acid and its γ-amide, NCA tyrosine, NCA serine, NCA L-threonine, NCA L-phenylalanine, NCA L-valine, NCA L-leucine, NCA L-isoleucine, NCA L-aniline, followed by polymerization.

An initiator is any chemical species capable of initiating a polymerization process, especially any nucleophilic species such as a primary, secondary or tertiary C1-C12 alkylamine, a C1-C12 alkyldiamine, a primary or secondary arylamine, an amino acid having one or more free amine functions, or a peptide having one or more free amine functions.

A poly(lysine)-b-poly(arginine) block polymer may be synthesized by the method described in the article ‘Linking poly-L-arginine to poly-DL-lysine using pentosidine linkages’, Abstracts of Papers, 225th ACS National Meeting, New Orleans, La., United States, Mar. 23-27, 2003.

It is also possible to prepare a backbone made up of the assembly of non-cationic amino acid units, and then to convert them, in a subsequent step, into cationic units. This is the case, for example, for the derivation of α,β-poly(N-2-hydroxyethyl)-DL-aspartamide (PHEA) and α,β-polyaspartylhydrazide with glycidyltrimethylammonium chloride (Giammona et al., Polymer International (Polym. Int.) 2000, Vol. 49, No. 1, pp. 93-98).

When the backbone further comprises additional monomers or polymers, a person skilled in the art will know how, on the basis of his or her general knowledge, to select the most appropriate method in accordance with the known rules of chemical reactivity.

Thus a polymer backbone of PEG-b-poly(amino acid) type may be prepared by the method described in patent U.S. Pat. No. 6,686,446, or in the article ‘Synthesis and Evaluation of Poly(Ethylene Glycol)-Polylysine Block Copolymers as Carriers for Gene Delivery’ by Vanderkerken, Journal of Bioactive and Compatible Polymers, Vol. 15, No. 2, 115-138 (2000).

In order to illustrate the synthesis of polyamino acid-poly(alkylene oxide)-polyamino acid triblocks, especially PLL-PEG-PLL, it is also possible to cite the article ‘Preparation of PLL-PEG-PLL and its application to DNA encapsulation’ of Yang et al., in Science in China, Series B, Chemistry (2006), 49(4), 357-362. Oligosaccharide-poly(lysine) polymers or dendrimers may be prepared by the method described in the article ‘Synthesis of an oligosaccharide-polylysine dendrimer with reducing sugar terminals leading to acquired immunodeficiency syndrome vaccine preparation’, in Journal of Polymer Science, part A. Polymer Chemistry, 2005, Vol. 43, No. 11, pp. 2195-2206.

The synthesis of polybutadiene-b-poly(lysine) polymers may be performed by the method described in ‘Synthesis and characterization of pH sensitive PB-P(lys) block copolymer assemblies’ in PMSE Preprints (2005), 93, pp. 198-199.

When the backbone further comprises vinyl monomers, it may be synthesized by the conventional methods of peptide sequence functionalization with, for example, a halogen, followed by the polymerization of vinyl monomers by controlled polymerization techniques, coupling for example ATRP as described by Klok in “Biological-synthetic hybrid block copolymers: combining the best from two worlds”, Journal of Polymer Science Part A: Polymer Chemistry 2005, Volume 43, Issue 1, pp. 1-17.

When the backbone further comprises saccharide, urethane or urea monomers, it may be prepared in a way similar to that described by Schlaad et al. in “Block copolymers with aminoacid sequences: Molecular chimeras of polypeptides and synthetic polymers”, The European Physical Journal E, Volume 10, No. 1, 2003, pp. 17 to 23.

The polymer backbone is preferably composed 100% of cationic amino acid monomers.

When it comprises additional monomers, in other words monomers other than the cationic amino acid monomers, said additional monomers may represent 0.1% to 50% by weight, especially 1% to 45% by weight, and even 2% to 25% by weight of the total weight of the monomers forming the backbone.

In this case the cationic amino acid monomers represent 50% to 99.9% by weight, preferably 55% to 99% by weight, and even 75% to 98% by weight of the total weight of monomers forming the backbone.

The polymer backbone preferably comprises amino acid monomers connected by amide linkages; this polymer backbone may be natural or synthetic in origin.

Preferably the polymer backbone is selected from poly(α-lysine) , poly(ε-lysine), poly(L-lysine), poly(D-lysine), poly(ornithine), poly(histidine), poly(arginine) or poly(ornithine-co-serine) polymers.

Preferably the polymer backbone has a molecular weight (Mw) of between 1000 and 5,000,000, especially between 2000 and 1,000,000 and preferably between 3000 and 100,000 g/mol.

The polymer according to the invention also comprises hydrophilic grafts bonded covalently to said polymer backbone.

The hydrophilic grafts are characterized in that they contain the repetition of at least two hydrophilic monomer units, i.e. units having a value for the base-ten logarithm of the apparent octan-1-ol/water partition coefficient, also called log p, of less than or equal to 2, for example between −10 and 1.8, preferably between −4 and 1.5, especially between −3 and 1, and even between −2.5 and 0.5.

The graft preferably contains the repetition of at least 5 identical or different hydrophilic units.

The log p values are known and are determined by a standard test which determines the concentration of the monomer in octan-1-ol and water.

The values may especially be calculated using the ACD (Advanced Chemistry Development) Solaris software V4.67; they may also be obtained from Exploring QSAR: hydrophobic, electronic and steric constants (ACS professional reference book, 1995). There is also an Internet site which provides estimated values (address: http://esc.syrres.com/interkow/kowdemo.htm).

Indicated below is the log p value determined using the ACD software for certain repeating units that may be present in the hydrophilic grafts.

| Unit | Fragmental log p value |
|---|---|
| —CH₂—O— | −0.246 |
| —CH₂—CH₂—O— | −0.136 |
| —CH₂—CH(OH)— | −0.493 |
| —CH₂—CH(OCH₃)— | 0.295 |
| —CH₂—CH₂—CH₂—CH₂—O— | 0.176 |
| —CH(OH)—CH(OH)—CH(OH)— | −2.476 |
| 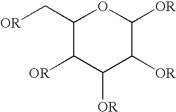 | log p less than 0 |
| in which two of the Rs | |
| represent a single bond, | |
| the 3 other Rs representing | |
| H | |
| 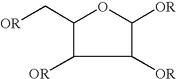 | log p less than 0 |
| in which two of the Rs | |
| represent a single bond, | |
| the 2 other Rs representing | |
| H | |
| 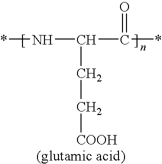 | −1.146 |
| 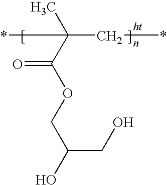 | −0.7038 |
| 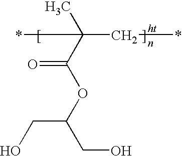 | −0.7532 |
| 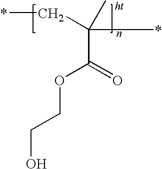 | 0.1214 |

Preferably the hydrophilic monomer units principally forming the hydrophilic graft are selected from the following units, and more preferably exclusively from these units:

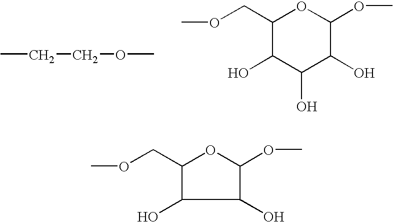

Very preferably the hydrophilic monomer units principally forming the hydrophilic graft are of formula —CH₂—CH₂—O—.

According to the invention the hydrophilic grafts are selected from polyalkylene oxides, and especially polyethylene oxide.

Preferably each hydrophilic graft possesses a number-average molecular mass (Mn) of between 500 g/mol and 100,000 g/mol, preferably between 550 g/mol and 50,000 g/mol, more preferably between 1000 and 40,000 g/mol.

The polymer according to the invention preferably has a degree of grafting of between 1% and 99%, especially between 2% and 50%, preferably between 3% and 45%, and more preferably between 4% and 35%, and even between 5% and 25%.

The degree of grafting for the purposes of the present description is the ratio (×100) between the number of cationic amino acid units grafted by a hydrophilic graft, and the total number of amino acid units (cationic units and, where appropriate, non-cationic units).

The hydrophilic grafts may be distributed on the polymer backbone randomly, alternatingly (1 graft for every 2, 3 or x cationic amino acid units), and even in blocks (no grafts on one part of the backbone, then all of the cationic amino acid units of part of the backbone grafted, for example). Preferably they are distributed randomly.

Preferably the polymer backbone has a molecular weight (Mw) of between 1000 and 5,000,000, especially between 2000 and 1,000,000, and each hydrophilic graft possesses a number-average molecular mass (Mn) of between 500 g/mol and 100,000 g/mol, preferably between 550 g/mol and 50,000 g/mol.

More preferably the polymer backbone has a molecular weight (Mw) of between 3000 and 100,000 g/mol, and each hydrophilic graft possesses a number-average molecular mass (Mn) of between 1000 and 40,000 g/mol.

The polymer according to the invention may be obtained in different ways.

In a first mode of preparation of the polymers according to the invention, it is possible in a first stage to prepare a functionalized polymer backbone, i.e. a polymer backbone carrying reactive chemical functions, and then, in a second stage, to graft the functionalized hydrophilic grafts containing the hydrophilic repeating units, by reacting the reactive functions carried by the grafts with the reactive functions carried by the polymer backbone. This technique is known to a person skilled in the art as ‘grafting to’.

The grafting reaction may be of any chemical kind.

For example, when the polymer backbone is composed solely of cationic amino acid units, it is possible to use the reactive primary, secondary and/or tertiary amine functions of said backbone to couple the hydrophilic graft, via any reaction of organic chemistry that is known to a person skilled in the art. Thus, for example, in the case of a poly(lysine) backbone, it is possible to use hydrophilic grafts terminated with a function capable of reacting with the primary amines of the lysine, for example a carboxylic acid function; the reaction is performed according to conventional coupling techniques such as an activated ester function such as N-hydroxysuccinimide esters.

The reaction may also be a reductive amination reaction, i.e. a coupling between an aldehyde functionalized graft and a primary amine function carried by the backbone; a Schiff base is formed which can be reduced to form an amine linkage.

When the backbone is composed of cationic amino acid units and non-cationic amino acid units, it is possible to use the same techniques as those described above. It is also possible, according to the techniques known to a person skilled in the art, to couple the hydrophilic grafts with non-cationic amino acid units. Thus, for example, in the case of a copolymer containing cationic amino acid units and serine units, it is possible to use the free hydroxyl functions of the serine (after protection of the cationic amino acid functions in the case of a primary amine such as lysine) to couple a hydrophilic graft terminated by a reactive function with the hydroxyl functions, for example a carboxylic acid function.

When the grafts are functionalized with (meth)acrylic groups at the chain end, and when the polymer backbone is functionalized with primary amines, it is also possible to contemplate a coupling reaction via a Michael addition reaction.

This is especially the case when the grafts, before polymerization on the backbone, are present in the form of macromonomers, i.e. prepolymers comprising—generally situated at at least one of their ends—a reactive group which allows said macromonomer to react with a function situated on the polymer backbone. Examples of such macromonomers include primarily poly(ethylene glycol) (meth)acrylates, or alkyl-poly(ethylene glycol) (meth)acrylates, especially methyl-poly(ethylene glycol) (meth)acrylates, also called methoxy-poly(ethylene glycol) or MPEG (meth)acrylates.

It is also possible to contemplate grafting via chemical reactions which are common knowledge in organic synthesis in intermediate steps, so as to functionalize the graft and/or the backbone such that a coupling reaction between them can take place.

Thus, for example, it is possible to functionalize a hydrophilic graft at the chain end with a lactone unit, such that it is subsequently able to react with a (primary) amine function of the backbone, it being possible for said lactone function to result from the lactonization of a carboxylic acid function, which itself may result from the oxidation of a hydroxyl group.

Mention may also be made of thiol, aldehyde, amine, succinimide, dichlorotriazine and maleic anhydride functions, which are able to react with maleimide functions (for the thiols), amine functions (aldehydes), aldehyde functions (amines), and primary amine functions, with regard to succinimide, dichlorotriazine and maleic anhydride functions.

In all the cases a person skilled in the art will know how to select the reactive function carried by the graft in accordance with the reactive function of the backbone, on the basis of his or her general knowledge.

By way of example, a polyalkylene glycol graft may be grafted onto the backbone, by virtue of coupling between its reduced terminal function, converted to an aldehyde, and a primary amine function carried by the polymer backbone. In this way a Schiff base is formed which can be reduced to form an amine linkage (reductive amination).

Another example consists in oxidizing the terminal OH function of the graft to an acid, using, for example, iodine, followed by a lactonization. Grafting is permitted by a subsequent reaction of the lactone function with an amine function of the backbone.

It should be specified that the grafts may, furthermore, be functionalized with a terminal function, for example, a thiol function, an aldehyde function, a succinic acid group, a biotin group, a dichlorotriazine, or a maleic anhydride group, these functions not being used to graft said graft onto the backbone.

In a second mode of preparation of the polymers according to the invention, it is possible in a first stage to prepare a functionalized polymeric backbone, i.e. a backbone carrying reactive chemical functions, which are capable of reacting with hydrophilic monomer units to give a graft polymer. This technique is known by a person skilled in the art as ‘grafting from’.

The monomer units may in this case either undergo polymerization directly via the cationic units, when the polymer backbone is composed solely of cationic amino acid units, or undergo polymerization onto other non-cationic amino acid units, when the starting materials are copolymers containing non-cationic amino acid units. Mention may be made, as an example, of the preparation of polylysine with polyglutamate grafts by a ‘grafting from’ technique as described in ‘Cylindrical polypeptide brushes’, Macromolecular Chemistry and Physics, (2005) 206(1), pp. 157-162.

In a third mode of preparation of the polymers of the invention, it is possible subsequently to functionalize a polymer according to the invention, for example to carry out the guanylation of a polylysine polymer grafted with dextran, as described in ‘Preparation of cationic comb-type copolymer having guanidino moieties and its interaction with DNAs’, Journal of Biomaterials Science, Polymer Edition (2004), 15(9), pp. 1099-1110.

Mention may also be made of the following article which illustrates the synthesis of polylysine grafted with poly(lactide-b-ethylene glycol): ‘Nanoparticulate DNA packaging using terpolymers of poly(lysine-g-(lactide-b-ethylene glycol)’ by Park et al., Bioconjugate Chemistry (2003), 14(2), pp. 311-319.

The polymers according to the invention therefore possess a polymer backbone which may result from the (co)polymerization of at least one amino acid having a cationic function, and comprise hydrophilic grafts bonded covalently to this backbone, over all or part of said backbone.

Preferably the backbone comprises at least the incorporation of an amide unit resulting, preferentially, from the (co)polymerization of amino acids.

The polymer according to the invention finds application most particularly in the cosmetic sector, especially in the hair sector.

The amount of polymer present in the compositions depends, of course, on the type of composition and on the desired properties, and may vary within a very wide range, generally between 0.01% and 30% by weight, preferably between 0.1% and 20% by weight, especially between 0.5% and 10% by weight, and even between 1% and 5% by weight, relative to the weight of the final cosmetic composition.

The compositions according to the invention may take any of the forms of formulation that are conventionally used for topical application, and especially may take the form of an aqueous, alcoholic or aqueous-alcoholic suspension or solution, or of an oily suspension or solution, or of a solution or a dispersion of the lotion or serum type, a milk-type emulsion of liquid or semi-liquid consistency, obtained by dispersing a fatty phase in an aqueous phase (O/W) or vice versa (W/O), or of a cream-type suspension or emulsion of soft consistency (O/W) or (W/O), or of an aqueous or anhydrous gel, of an ointment, of a loose or compacted powder to be used as it is or to be incorporated in an excipient, or of any other cosmetic form.

These compositions may be packaged, especially in pump flasks or in aerosol containers, in order to provide for application of the composition in vaporized form or in the form of a foam. Packaging forms of this kind are appropriate, for example, when the desire is to obtain a spray or a mousse for hair treatment. The compositions in accordance with the invention may also take the form of creams, gels, emulsions, lotions or waxes. When the composition according to the invention is packaged in aerosol form with the aim of obtaining a lacquer or a foam, it comprises at least one propellant.

Besides said polymers, the cosmetic compositions according to the invention comprise a cosmetically acceptable medium, in other words a medium which is compatible with keratin materials, especially the hair.

Said cosmetically acceptable medium comprises at least one cosmetic ingredient selected from propellants; carbon oils; silicone oils; C8-C40 alcohols, C8-C40 esters, C8-C40 acids; nonionic surfactants, cationic surfactants, anionic surfactants, amphoteric surfactants, zwitterionic surfactants; sunscreens; hydrating agents; anti-dandruff agents; antioxidants; reducing agents; oxidation bases, couplers, oxidizing agents, direct dyes; hair straightening agents; pearlizing agents and opacifiers; plasticizers or coalescers; hydroxy acids; pigments; fillers; silicones; and thickeners.

Said medium may of course comprise a plurality of cosmetic ingredients featuring in the list above.

The propellants may be present at a concentration of between 5% and 90% by weight relative to the total weight of the composition in the aerosol device, and more particularly at a concentration of between 10% and 60% by weight. The propellant may be selected especially from volatile hydrocarbons such as n-butane, propane, isobutane, pentane, a chlorinated and/or fluorinated hydrocarbon; carbon dioxide, nitrous oxide, dimethyl ether (DME), nitrogen, compressed air, and mixtures thereof.

The carbon oils, especially hydrocarbon oils, and/or the silicone oils may be present at from 0.01% to 20% by weight, especially 0.02% to 10% by weight, relative to the total weight of the composition. It is possible especially to mention hydrogenated or unhydrogenated plant, animal or mineral oils, saturated or unsaturated, linear or branched, cyclic or aliphatic, synthetic hydrocarbon oils, such as, for example, poly-alpha-olefins, more particularly polydecenes and polyisobutenes; water-soluble or water-insoluble, volatile or non-volatile silicone oils with or without organic modification; fluoro oils or perfluoro oils; and mixtures thereof.

The alcohols, the esters and the acids, having 8 to 40 carbon atoms, may be present at from 0.01% to 50% by weight, especially 0.1% to 20% by weight, relative to the total weight of the composition.

Mention may be made in particular of C12-C32, more particularly C12-C26, linear- or branched-chain fatty alcohols, and more particularly cetyl alcohol, stearyl alcohol, cetylstearyl alcohol, isostearyl alcohol, octyldodecanol, 2-butyloctanol, 2-hexyldecanol, 2-undecylpentadecanol, oleyl alcohol or linoleyl alcohol.

Mention may also be made of C8-C40, especially C16-C20, fatty alcohols which are alkoxylated, especially ethoxylated, preferably containing 10 to 50 moles of ethylene oxide and/or propylene oxide, such as oleth-12, ceteareth-12 and ceteareth-20, propoxylated stearyl alcohol especially containing 15 moles of propylene oxide, ethoxylated lauryl alcohol, especially containing more than 7 oxyethylene groups, and mixtures thereof.

Mention may also be made of C16-C40 linear- or branched-chain fatty acids, and especially 18-methyl-eicosanoic acid, copra oil acid or hydrogenated copra oil acid; stearic acid, lauric acid, palmitic acid and oleic acid, behenic acid, and mixtures thereof.

Mention may also be made of C16-C40 linear- or branched-chain fatty esters, such as the esters of polyols derived from fatty acids containing 8 to 30 carbon atoms, and their alkoxylated and especially ethoxylated derivatives, the polyols being preferably selected from sugars, C2-C6 alkylene glycols, glycerol, polyglycerols, sorbitol, sorbitan, polyethylene glycols, polypropylene glycols and mixtures thereof.

The alkoxylated polyol esters may contain 1 to 20 oxyalkylene groups, especially oxyethylene groups.

Glycerol esters include monoglycerides such as glyceryl oleate, glyceryl linoleate, glyceryl laurate, and mixtures thereof.

Polyglycerol esters include diglyceryl monoisostearate, diglyceryl oleate, triglyceryl monooleate, diglyceryl distearate, pentaglyceryl tristearate, and mixtures thereof.

An ethoxylated glycerol ester includes ethoxylated glycerol stearate, especially containing 20 oxyethylene units.

Sorbitan esters include, for example, sorbitan stearate, sorbitan laurate; sorbitan palmitate, sorbitan tristearate, sorbitan oleate and sorbitan trioleate.

Ethoxylated sorbitan esters include polysorbates, for example Polysorbate 21; and mixtures thereof.

Sugar esters include those derived from the following sugars: sucrose, glucose, fructose, mannose, galactose, arabinose, xylose, maltose, cellibiose, lactose, trehalose, raffinose, gentianose. Mention may be made, for example, of sucrose cocoate, sucrose monooctanoate, sucrose monodecanoate, sucrose monolaurate, sucrose monomyristate, sucrose monopalmitate, sucrose monostearate, sucrose monooleate, sucrose monolinoleate, sucrose dioleate, sucrose dipalmitate, sucrose distearate, sucrose dilaurate, sucrose dilinoleate, sucrose tristearate, octylglucofuranoside esters, galactolipids, and mixtures thereof.

Mention may also be made of synthetic esters, especially those of formula Ra-COORb in which Ra represents the residue of an acid containing 8 to 29 carbon atoms, and Rb represents a branched or unbranched hydrocarbon chain containing 3 to 30 carbon atoms, such as for example, purcellin oil, isononyl isononanoate, isopropyl myristate, 2-ethylhexyl palmitate, 2-octyldodecyl stearate, 2-octyldodecyl erucate, isostearyl isostearate; hydroxy esters such as isostearyl lactate, octyl hydroxystearate, octadodecyl hydroxystearate, diisostearyl malate, triisocetyl citrate, and the heptanoates, octanoates and decanoates of fatty alcohols.

The nonionic, cationic, anionic, amphoteric or zwitterionic surfactants, and also mixtures thereof, may be present at from 0.01% to 50% by weight, especially 0.05% to 40% by weight, and even 0.1% to 30% by weight, relative to the total weight of the composition.

The anionic surfactants that can be used, alone or in mixtures, include alkali metal salts, especially sodium salts, ammonium salts, amine salts, amino alcohol salts and magnesium salts, of the following compounds: alkyl sulphates, alkyl ether sulphates, alkylamidoether sulphates, monoglyceride sulphates, alkyl glycerylsulphonates, alkylsulphonates, alkyl phosphates, alkylamide sulphonates, alkylarylsulphonates, alpha-olefinsulphonates, paraffinsulphonates, alkyl sulphosuccinates, alkyl ether sulphosuccinates, alkylamide sulphosuccinates, alkyl sulphosuccinamates, alkyl sulphoacetates, alkyl ether phosphates, acylisethionates, N-acyltaurates, N-acylamino acids such as N-acylsarcosinates and N-acylglutamates. It is also possible to use salts of fatty acids such as the salts of undecenylic, oleic, ricinoleic, palmitic and stearic acids, or of copra oil acid or hydrogenated copra oil acid, or else of acylhydroxy acids such as the acyl-lactylates. It is also possible to use weakly anionic surfactants, such as alkyl-D-galactosiduronic acids and their salts, or polyalkoxylated ether carboxylic acids and their salts. The alkyl or acyl radical of the various surfactants set out above has preferably 8 to 22 carbon atoms.

The nonionic surfactants include alcohols, alpha-diols, alkylphenols or polyethoxylated, polypropoxylated or polyglycerolated fatty acids, having a fatty chain containing 8 to 22 carbon atoms, it being possible for the number of ethylene or propylene oxide groups to range from 2 to 50 and that of glycerol especially from 2 to 30. Mention may also be made of copolymers of propylene and ethylene oxide, polyethoxylated fatty amides or amines having preferably 2 to 30 moles of ethylene oxide, polyglycerolated fatty amides containing on average 1 to 5 glycerol groups, polyglycerolated diglycolamides, optionally ethoxylated sorbitan fatty acid esters, sucrose fatty acid esters, optionally alkoxylated alkylpolyglycosides, alkylglucoside esters, N-alkylglucamine derivatives and amine oxides.

Amphoteric or zwitterionic surfactants include the aliphatic secondary or tertiary amine derivatives in which the aliphatic radical is a linear or branched chain containing 8 to 22 carbon atoms and containing at least one water-solubilizing anionic group (for example carboxylate, sulphonate, sulphate, phosphate or phosphonate), such as, for example, alkylbetaines, alkylaminocarboxylates, sulphobetaines, alkylamidoalkylbetaines, alkylamidoalkylsulphobetaines, and imidazolium derivatives, especially those of amphocarboxyglycinate or amphocarboxypropionate.

The cationic surfactants include the salts of optionally polyalkoxylated and/or quaternized fatty amines, the esters of fatty acids and amino alcohols which are optionally polyalkoxylated and/or quaternized, quaternary ammonium salts such as tetraalkylammonium, alkylamidoalkyltrialkylammonium, trialkylbenzylammonium, trialkylhydroxyalkylammonium, dialkylamidoalkyldimethylammonium and alkylpyridinium chlorides or bromides, and imidazolium derivatives.

The sunscreen agents may be present at from 0.01% to 20% by weight, especially 0.5% to 10% by weight, relative to the total weight of the composition. These agents may be active in WA and/or UVB (absorbers), and may be fat-soluble or water-soluble. They may especially be selected from salicylic derivatives, camphor derivatives; triazine derivatives; benzophenone derivatives; diphenylacrylate derivatives, benzimidazole derivatives, bisbenzoxazolyl derivatives; derivatives of p-aminobenzoic acid; sunscreen polymers and sunscreen silicones.

The hydrating agents may be present at from 0.01% to 20% by weight, especially 0.1% to 7% by weight, relative to the total weight of the composition. Mention may be made in particular of polyols, sugars and proteins, and in particular glycerol, sorbitol, D-panthenol, mannitol; fructose, galactose, sucrose and N-acetylglucosamine.

The anti-dandruff agents may be present at from 0.001% to 20% by weight, especially 0.01% to 10% by weight, relative to the total weight of the composition, preferably 0.1% to 5% by weight. The following may be mentioned especially:

(a) pyridinethione salts, especially the calcium, magnesium, barium, strontium, zinc, cadmium, tin and zirconium salts.

(b) derivatives of 1-hydroxy-2-pyrrolidone of formula

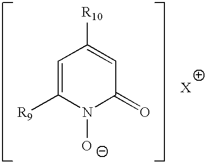

and its salts,
in which:

R₉ represents a C1-C17 alkyl group, a C2-C17 alkenyl group, a C5-C8 cycloalkyl group, a C7-C9 bicycloalkyl group; a cycloalkyl(alkyl) group, an aryl group, an aralkyl group with a C1-C4 alkyl, an arylalkenyl group with a C2-C4 alkenyl, aryloxyalkyl or arylmercaptoalkyl with a C1-C4 alkyl, a furylalkenyl group with a C2-C4 alkenyl, a C1-C4 alkoxy group, a nitro group, a cyano group or a halogen atom;

R₁₀ represents a hydrogen atom, a C1-C4 alkyl group, a C2-C4 alkenyl group, a halogen atom, a phenyl group, a benzyl group;

X represents an organic base, an alkali metal or alkaline earth metal ion or an ammonium ion.

(c) 2,2′-dithiobis(pyridine N-oxide) of formula:

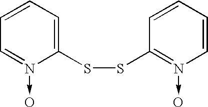

and also its inorganic salts, for example its magnesium sulphate salt.

(d) trihalocarbamides of formula:

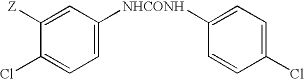

in which Z represents a halogen atom, especially Cl, or a C1-C4 trihaloalkyl group such as CF3;

(e) azole compounds such as climbazole, ketoconazole, clotrimazole, econazole, isoconazole and miconazole; antifungal polymers such as amphotericin B or nystatin; selenium sulphide; sulphur in its various forms, cadmium sulphide, allantoin; coal tar or wood tar and derivatives thereof, more particularly cade oil; salicylic acid, undecylenic acid, fumaric acid; allylamines such as terbinafine.

The antioxidants may be present at from 0.05% to 1.5% by weight, relative to the total weight of the composition. Mention may be made in particular of polyphenols, vitamin E, dehydroascorbic acid, hydroquinone, 2-methylhydroquinone, tert-butylhydroquinone and homogentisic acid.

The reducing agents may be present at from 0.1% to 30% by weight, especially 0.5% to 20% by weight, relative to the total weight of the composition. Mention may be made especially of sulphites, bisulphites, alkylphosphines and thiols. Very particularly mention may be made of thioglycolic acid or thiolactic acid and their ester and amide derivatives, especially glycerol monothioglycolate; cysteamine and its C1-C4 acyl derivatives such as N-acetylcysteamine or N-propionylcysteamine; cysteine, N-acetylcysteine, thiomalic acid, panthetein, 2,3-dimercaptosuccinic acid; sulphites or bisulphites of an alkali metal or alkaline earth metal, N-(mercaptoalkyl)-ω-hydroxyalkylamides, N-mono- or N,N-dialkylmercapto-4-butyramides, aminomercaptoalkylamides, derivatives of N-(mercaptoalkyl)succinamic acids and N-(mercaptoalkyl)succinimides, alkylaminomercaptoalkylamides; 2-hydroxypropyl thioglycolate, (2-hydroxy-1-methyl)ethyl thioglycolate and the azeotropic mixture thereof; mercaptoalkylamino amides; N-mercaptoalkylalkanediamides, and the derivatives of formamidine-sulphinic acid; ascorbic acid, its salts and its esters; erythorbic acid, its salts and its esters; and sulphinates such as sodium hydroxymethanesulphinate.

The products employed for colouring the hair may be selected from oxidation bases, couplers, oxidizing agents, direct dyes, and mixtures thereof.

As an oxidation base, mention may be made especially of para-phenylenediamines, bisphenylalkylenediamines, para-aminophenols, ortho-aminophenols and heterocyclic bases, among which mention may be made, by way of example, of pyridine derivatives, pyrimidine derivatives and pyrazole derivatives.

Among the couplers, mention may be made especially of meta-phenylenediamines, meta-aminophenols, meta-diphenols, naphthalene couplers and heterocyclic couplers.

The oxidation bases may be present in an amount of between 0.001% to 10% by weight, preferably from 0.005% to 6% by weight, of the total weight of the composition.

The couplers may be present in an amount of between 0.001% and 10% by weight, preferably from 0.005% to 6% by weight, of the total weight of the composition.

The oxidizing agents may be selected from hydrogen peroxide or alkali metal bromides, or else from hydrogen peroxide, urea peroxide, alkali metal bromides, persalts such as perborates and persulphates, and also enzymes, including peroxidases, 2-electron oxidoreductases such as uricases, and 4-electron oxygenases such as laccases.

The amount of oxidizing agent may be between 1% and 40% by weight, preferably between 1% and 20% by weight, relative to the weight of the composition.

The direct dyes may be selected from cationic or nonionic species, and especially from nitrobenzene dyes, azo, azomethine, methine, tetraazapentamethine, azine, anthraquinone, quinone, naphthoquinone, benzoquinone, phenothiazine, indigoid, xanthene, phenanthridine, phthalocyanine, triarylmethane and indoamine dyes, and natural dyes, alone or in mixtures. The amount of direct dye may be between 0.001% to 20% by weight, preferably from 0.01% to 10% by weight, relative to the total weight of the composition.

The hair straightening agents may be present at from 0.01% to 3.5% by weight, especially 0.05% to 1.5% by weight, relative to the total weight of the composition. Mention may be made especially of hydroxides, selected preferably from alkali metal or alkaline earth metal hydroxides, transition metal hydroxides or organic hydroxides, such as sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and quaternary ammonium hydroxides.

The pearlizing agents and opacifiers may be present at from 0.01% to 3% by weight, especially 0.05% to 2.5% by weight, relative to the total weight of the composition. Mention may be made especially of sodium or potassium palmitates, sodium or potassium stearates or hydroxystearates, and ethylene glycol mono- or distearate.

The plasticizers or coalescers may be present at from 0.1% to 25% by weight, especially 1% to 10% by weight, relative to the total weight of the composition. Mention may be made especially, alone or in a mixture, of glycols and their derivatives; glycerol esters, such as glycerol diacetate or triacetate; propylene glycol derivatives; esters of acids, especially of carboxylic acids, such as citrates, phthalates, adipates, carbonates, tartrates, phosphates and sebacates; ethoxylated derivatives such as ethoxylated oils, especially vegetable oils such as castor oil; and ethoxylated silicone oils.

The hydroxy acids may be present at from 1% to 10% by weight, especially 2% to 5% by weight, relative to the total weight of the composition. Mention may be made in particular of monocarboxylic or polycarboxylic acids containing one or more hydroxy functions; the hydroxy acid is preferably an alpha-hydroxy acid. More particularly mention may be made of citric, lactic, methyllactic, phenyllactic, malic, mandelic, glycolic, tartronic, tartaric, gluconic, benzylic and 2-hydroxycaprylic acid.

The pigments and fillers may be present at from 0.01% to 50% by weight, especially 0.02% to 30% by weight, relative to the total weight of the composition. Mention may be made especially of organic or inorganic fillers of any form: platelet-shaped, spherical or oblong. Mention may be made more particularly of titanium oxide, talc, mica, silica, kaolin, polyamide (nylon) powders, polyalanine powders and polyethylene powders, powders of tetrafluoroethylene polymers (Teflon), lauroyllysine, starch, boron nitride, hollow polymeric microspheres such as those of polyvinylidene chloride/acrylonitrile, like Expancel, or of acrylic acid copolymers (Polytrap); silicone resin microbeads (Tospearls), particles of elastomeric polyorganosiloxanes, precipitated calcium carbonate, magnesium carbonate and bicarbonate, hydroxyapatite, hollow silica microspheres; glass or ceramic microcapsules, metal soaps derived from organic carboxylic acids having 8 to 22 carbon atoms, preferably 12 to 18 carbon atoms, for example zinc stearate, magnesium stearate or lithium stearate, zinc laurate and magnesium myristate.

The silicones may be volatile or non-volatile; mention may be made especially of modified or unmodified polyorganosiloxanes, namely polyorganosiloxane oils, gums and resins, as they are or in the form of solutions in organic solvents, or in the form of emulsions or microemulsions.

Mention may be made more particularly of the following, alone or in a mixture:

(a) volatile silicones, possessing a boiling point of between 60° C. and 260° C., which may be cyclic silicones containing 3 to 7 silicon atoms and preferably 4 to 5. Examples include octamethylcyclotetrasiloxane, decamethylcyclopentasiloxane and cyclocopolymers of the dimethylsiloxane/methylalkylsiloxane type, such as dimethylsiloxane/methyloctylsiloxane;

(b) polyalkylsiloxanes, and primarily polydimethylsiloxanes, especially linear polyalkylsiloxanes containing terminal trimethylsilyl groups; DC 200 oils from Dow Corning; PDMS containing terminal hydroxydimethylsilyl groups;

(c) polyarylsiloxanes;

(d) polyalkylarylsiloxanes, and especially linear or branched polymethylphenylsiloxanes, polydimethylmethylphenylsiloxanes and polydimethyldiphenylsiloxanes;

(e) silicone gums; these are polydiorganosiloxanes with a molecular mass of between 200,000 and 5,000,000 which can be used alone or in a mixture in a solvent selected from volatile silicones, polydimethylsiloxane (PDMS) oils, polyphenylmethylsiloxane (PPMS) oils, isoparaffins, methylene chloride, pentane, dodecane, tridecane, tetradecane or mixtures thereof. Examples include the following compounds: polydimethylsiloxanes, poly[(dimethylsiloxane)/(methylvinylsiloxane)]s, poly[(dimethylsiloxane)/(diphenylsiloxane)]s, poly[(dimethylsiloxane)/(phenylmethylsiloxane)]s, and poly[(dimethylsiloxane)/(diphenylsiloxane)/(methylvinylsiloxane)]s. Mention may also be made of the following mixtures:

mixtures formed from a polydimethylsiloxane which is hydroxylated at the chain end (dimethiconol) and a cyclic polydimethylsiloxane, such as the product Q2 1401 from Dow Corning;

mixtures formed from a polydimethylsiloxane gum with a cyclic silicone;

mixtures of two PDMS of different viscosities, especially a PDMS gum and a PDMS oil;

(f) silicone resins; preferably crosslinked siloxane systems containing the units R2SiO2/2, RSiO3/2 and SiO4/2, in which R represents a hydrocarbon group possessing 1 to 6 carbon atoms or a phenyl group;

(g) organically modified polyorganosiloxanes, in other words silicones as defined above, containing in their general structure one or more organofunctional groups attached directly to the siloxane chain or attached via a hydrocarbon radical; mention may be made of the silicones containing:

polyethyleneoxy and/or polypropyleneoxy groups, optionally containing alkyl groups, such as dimethicone copolyols and alkyl(C12)methicone copolyol;

(per)fluorinated groups such as trifluoroalkyl groups;

hydroxyacylamino groups;

thiol groups;

substituted or unsubstituted amine groups.

Substituted amine groups are, in particular, C1-C4 aminoalkyl groups or (C1-C4)aminoalkyl (C1-C4)aminoalkyl groups. Use is made more particularly of the silicones named amodimethicone and trimethylsilylamodimethicone in accordance with the CTFA nomenclature;

carboxylate groups;

hydroxyl groups, such as hydroxyalkyl-functional polyorganosiloxanes;

alkoxy groups containing at least 12 carbon atoms;

acyloxyalkyl groups containing at least 12 carbon atoms;

quaternary ammonium groups;

amphoteric or betaine groups;

bisulphite groups;

(h) block copolymers having a linear polysiloxane-polyalkylene block as a repeating unit;

(i) graft silicone polymers with a non-silicone organic backbone, composed of an organic main chain formed from organic monomers not containing silicone, on which is grafted, inside said chain and also, optionally, at at least one of its ends, at least one polysiloxane macromonomer;

(j) graft silicone polymers with a polysiloxane backbone grafted with non-silicone organic monomers, composed of a polysiloxane main chain, on which is grafted, inside said chain and also, optionally, at at least one of its ends, at least one organic macromonomer not containing silicone.

The thickeners may be present at from 0.01% to 10% by weight, especially from 0.1% to 5% by weight, relative to the total weight of the composition. They may be selected from cellulose, cellulose derivatives, acrylic thickening polymers (Carbopol), alginates, gums such as xanthan gum, guar gum, carob gum or gum arabic, or else polyethylene glycols, bentonites and montmorillonites.

A person skilled in the art will ensure that the ingredients forming the cosmetic composition, and their amounts, are selected such that they are not detrimental to the properties of the compositions of the present invention.

Furthermore, the composition may comprise water, one or more C1-C6 alcohols, alone or in a mixture with water, and especially a water/ethanol, water/isopropanol or water/benzyl alcohol mixture.

It may also comprise polymers, especially polymers which are water-soluble or soluble in carbon oils and/or silicone oils; they may be present at from 0.01% to 20% by weight, especially 0.1% to 10% by weight, relative to the total weight of the composition.

The composition may for example comprise a film-forming polymer, which is a polymer capable of forming—by itself or in the presence of an auxiliary film-forming agent—a continuous film which is adherent to a substrate, especially to keratin materials. The film-forming polymers that can be used in the composition of the present invention include synthetic polymers, of free-radical or polycondensate type, polymers of natural origin and mixtures thereof, more particularly acrylic polymers, polyurethanes, polyesters, polyamides, polyureas and cellulosic polymers such as nitrocellulose.

The polymer may also be selected from cationic polymers of polyamine, polyaminoamide and polyquaternary ammonium type, and especially:

(1) unquaternized or quaternized dialkylaminoalkyl acrylate- or methacrylate-vinylpyrrolidone copolymers, such as the polymers described in FR2077143 and FR2393573.

(2) Cellulose ether derivatives containing quaternary ammonium groups, described in FR1492597.

(3) Cationic cellulose derivatives such as copolymers of cellulose or derivatives of cellulose grafted with a water-soluble quaternary ammonium monomer, which are described especially in U.S. Pat. No. 4,131,576, such as hydroxyalkylcelluloses, for instance hydroxymethyl-, hydroxyethyl- or hydroxypropylcelluloses, grafted especially with a methacryloylethyltrimethylammonium, methacrylamidopropyltrimethylammonium or dimethyldiallylammonium salt.

(4) Cationic polysaccharides and especially guar gums, described more particularly in U.S. Pat. Nos. 3,589,578 and 4,031,307.

(5) Polymers composed of piperazinyl units and divalent straight-chain or branched-chain alkylene or hydroxyalkylene radicals, optionally interrupted by oxygen, sulphur or nitrogen atoms or by aromatic or heterocyclic rings, and also the oxidation and/or quaternization products of these polymers. Polymers of this kind are described especially in FR2162025 and FR2280361.

(6) Water-soluble polyaminoamides prepared more particularly by polycondensation of an acid compound with a polyamine; these polyaminoamides may be crosslinked with an epihalohydrin, a diepoxide, a dianhydride, an unsaturated dianhydride, a diunsaturated derivative, a bishalohydrin, a bisazetidinium, a bishaloacyldiamine, a bisalkyl halide or else with an oligomer resulting from the reaction of a difunctional compound reactive towards a bishalohydrin, a bisazetidinium, a bishaloacyldiamine, a bisalkyl halide, an epihalohydrin, a diepoxide or a diunsaturated derivative, the crosslinking agent being used in proportions ranging from 0.025 to 0.35 mol % per amine group of the polyaminoamide; these polyaminoamides may be alkylated or they contain one or more quaternized tertiary amine functions. Polymers of this kind are especially described in FR2252840 and FR2368508. The polyaminoamide derivatives result from the condensation of polyalkylenepolyamines with polycarboxylic acids, followed by alkylation with difunctional agents. Mention may be made, for example, of the adipic acid-diacylaminohydroxyalkyldialkylenetriamine polymers in which the alkyl radical contains 1 to 4 carbon atoms and is preferably methyl, ethyl or propyl. Polymers of this kind are especially described in FR1583363.

(8) Polymers obtained by reacting a polyalkylenepolyamine containing two primary amine groups and at least one secondary amine group with a dicarboxylic acid selected from diglycolic acid and saturated aliphatic dicarboxylic acids having 3 to 8 carbon atoms, the molar ratio between the polyalkylenepolyamine and the dicarboxylic acid being between 0.8:1 and 1.4:1, the resulting polyaminoamide being reacted with epichlorohydrin in a molar ratio of epichlorohydrin relative to the secondary amine group of the polyaminoamide of between 0.5:1 and 1.8:1. Polymers of this kind are especially described in U.S. Pat. Nos. 3227615 and2,961,347.

(9) Methyldiallylamine or dimethyldiallylammonium cyclopolymers, especially those described in FR2080759 and in FR2190406.

(10) The quaternary diammonium polymers described in French Patents 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

(11) Quaternary polyammonium polymers, especially described in EP122324.

(12) Quaternary vinylpyrrolidone and vinylimidazole polymers such as, for example, the products sold under the names Luviquat FC 905, Luviquat FC 550 and Luviquat FC 370 by BASF.

(13) Polyamines such as Polyquart H, sold by Henkel, referenced under the name Polyethylene Glycol Tallow Polyamine in the CTFA dictionary.

(14) Crosslinked polymers of methacryloyloxyethyltrimethylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, more particularly methylenebisacrylamide. It is possible more particularly to use a crosslinked acrylamide/methacryloyloxyethyltrimethylammonium chloride (20/80 by weight) copolymer in the form of a dispersion containing 50% by weight of said copolymer in mineral oil.

Other cationic polymers which can be used in the context of the invention are polyalkyleneimines, more particularly polyethyleneimines, polymers containing vinylpyridine or vinylpyridinium units, condensates of polyamines and epichlorohydrin, quaternary polyureylenes, and chitin derivatives. Preference is given to cellulose ether derivatives, containing quaternary ammonium groups, cationic polysaccharides and especially guar gum, and methyldiallylamine or dimethyldiallylammonium cyclopolymers.

Mention may also be made of cationic fixative film-forming polymers selected from polymers containing primary, secondary, tertiary and/or quaternary amine groups forming part of the polymer chain or directly connected thereto, and having a molecular weight of between 500 and about 5,000,000 and preferably between 1000 and 3,000,000. Mention may be made especially of the following:

(1) polymers containing the following units:

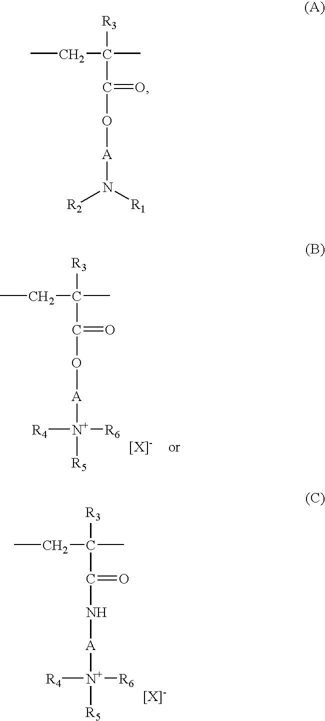

in which:

R₃ denotes a hydrogen atom or a CH₃ radical;

A is a divalent linear or branched alkylene group containing 1 to 6 carbon atoms or a hydroxyalkylene group containing 1 to 4 carbon atoms;

R₁ and R₂, which are identical or different, represent a hydrogen atom or an alkyl group having 1 to 6 carbon atoms;

R₄, R₅ and R₆, which are identical or different, represent an alkyl group having 1 to 18 carbon atoms or a benzyl radical;

X denotes a methosulphate anion or a halide such as chloride, iodide or bromide.

These polymers of class (1) may further contain one or more units deriving from comonomers which may be selected from the class of acrylamides, methacrylamides, diacetone acrylamides, acrylamides and methacrylamides substituted on the nitrogen by lower (C1-C4) alkyl groups, groups derived from acrylic or methacrylic acids or their esters, vinyllactams such as vinylpyrrolidone or vinylcaprolactam, and vinyl esters. Accordingly the polymers of class (1) include:

copolymers of acrylamide and dimethylaminoethyl methacrylate quaternized with dimethyl sulphate or with a dimethyl halide;

copolymers of acrylamide and methacryloyloxyethyltrimethylammonium chloride;

the copolymer of acrylamide and methacryloyloxyethyltrimethylammonium methosulphate;

quaternized or unquaternized vinylpyrrolidone/dialkylaminoalkyl acrylate or methacrylate copolymers;

fatty-chain polymers containing a vinylpyrrolidone unit;

dimethylaminoethyl methacrylate/vinylcaprolactam/vinylpyrrolidone terpolymers;

quaternized vinylpyrrolidone/dimethylaminopropylmethacrylamide copolymers;

(2) cationic polysaccharides, preferably containing quaternary ammonium, such as those described in U.S. Pat. Nos. 3,589,578 and 4,031,307, such as guar gums containing cationic trialkylammonium groups. Products of this kind are sold especially under the tradenames Jaguar C13 S, Jaguar C 15 and Jaguar C by Meyhall;

(3) quaternary vinylpyrrolidone and vinylimidazole copolymers;

(4) chitosans and salts thereof; the salts which can be used are more particularly the acetate, lactate, glutamate, gluconate or pyrrolidonecarboxylate of chitosan. These compounds include chitosan having a degree of deacetylation of 90.5% by weight, sold under the name Kytan Brut Standard by Aber Technologies, and chitosan pyrrolidonecarboxylate, sold under the name Kytamer® PC by Amerchol.

Mention may also be made of amphoteric fixative polymers selected from polymers containing units B and C distributed randomly in the polymer chain, where B denotes a unit deriving from a monomer containing at least one basic nitrogen atom and C denotes a unit deriving from an acidic monomer containing one or more carboxylic or sulphonic groups, or else B and C may denote groups deriving from zwitterionic carboxybetaine or sulphobetaine monomers; B and C may also denote a cationic polymer chain containing primary, secondary, tertiary or quaternary amine groups, at least one of the amine groups in said chain carrying a carboxylic or sulphonic group connected via a hydrocarbon group, or else B and C form part of a chain of a polymer containing an ethylene-α,β-dicarboxylic unit in which one of the carboxyl groups has been reacted with a polyamine containing one or more primary or secondary amine groups. Preferred amphoteric fixative polymers are selected from the following polymers:

(1) copolymers containing acidic vinyl units and containing basic vinyl units, such as those resulting from the copolymerization of a monomer derived from a vinyl compound carrying a carboxylic group, such as, more particularly, acrylic acid, methacrylic acid, maleic acid, alpha-chloroacrylic acid, and a basic monomer derived from a substituted vinyl compound containing at least one basic atom, such as, more particularly, dialkylaminoalkyl acrylates and methacrylates and dialkylaminoalkylmethacrylamides and -acrylamides.

(2) Polymers containing units deriving:

a) from at least one monomer selected from acrylamides or methacrylamides substituted on the nitrogen atom by an alkyl group,

b) from at least one acidic comonomer containing one or more reactive carboxylic groups, and

c) from at least one basic comonomer such as esters, having primary, secondary, tertiary and quaternary amine substituents, of acrylic and methacrylic acids, and the quaternization product of dimethylaminoethyl methacrylate with dimethyl or diethyl sulphate.

The N-substituted acrylamides or methacrylamides which are more particularly preferred are the compounds in which the alkyl groups contain 2 to 12 carbon atoms, and more particularly N-ethylacrylamide, N-tert-butylacrylamide, N-tert-octylacrylamide, N-octylacrylamide, N-decylacrylamide, N-dodecylacrylamide, and the corresponding methacrylamides.

The acidic comonomers are selected more particularly from acrylic, methacrylic, crotonic, itaconic, maleic and fumaric acids and also the alkyl monoesters having 1 to 4 carbon atoms of the acids or of maleic or fumaric anhydride.

Preferred basic comonomers are aminoethyl, butylaminoethyl, N,N′-dimethylaminoethyl and N-tert-butylaminoethyl methacrylates.

Preferably the composition according to the invention may take the form of a thickened or unthickened lotion, a thickened or unthickened cream, a gel, a foam or mousse, or any other appropriate form. It may where appropriate be packaged in a pump flask or in an aerosol container. Preferably it takes the form of a thickened or unthickened lotion.

The cosmetic composition according to the invention may take the form of a hair product, especially a hair care, hair cleansing, hair make-up, hair styling, hair shaping or hair colouring product. The composition finds particularly advantageous application, especially, in the hair management field, especially for holding the style or shaping the hair, or else for the care, cosmetic treatment or cleansing of the hair. The hair management compositions are preferably shampoos, conditioners, care or styling gels, care lotions or creams, conditioning products, setting lotions, blow-drying lotions, fixative and styling compositions such as lacquers or sprays; hair restructuring lotion; anti-hair loss lotion or gel, anti-parasitic shampoo, anti-dandruff lotion or shampoo, or anti-seborrhoeic treatment shampoo. The lotions may be packaged in various forms, especially in vaporizers, pump flasks or aerosol containers, for providing for application of the composition in vaporized form or in the form of a foam or mousse.

The composition may especially take the form of a hair colouring product, especially an oxidation dyeing product, where appropriate in the form of a colouring shampoo; or the form of a perming, straightening or bleaching composition, or else the form of a rinsing composition, for application before or after a colouring, bleaching, perming or straightening operation, or else between the two steps of a perming or straightening procedure.

Even more particularly, the composition according to the invention finds advantageous application for the care and cosmetic treatment, especially the protection, of the hair, more particularly of weakened and/or damaged hair, by means, for example, of chemical or mechanical treatments; the polymers according to the invention can be used especially in aftertreatment, after a hair colouring, bleaching or straightening step.

The reason for this is that it is known that the problem of the technique of permanent waving known to date is that its application to the hair may over the long term induce a deterioration in the quality of the hair, which is manifested in a decrease in its cosmetic properties, such as its gloss, and in a degradation of its mechanical properties, more particularly a degradation of its mechanical strength, and even in an increase in its porosity. The weakened hair may become fragile, especially in subsequent treatments such as blowdrying.

The invention accordingly provides a cosmetic treatment method, especially for making up, caring for, cleansing, colouring or shaping keratin materials, especially the hair, which comprises applying to said materials a cosmetic composition comprising at least one polymer as defined above.

The cosmetic treatment method in question is preferably a method for the care and/or cleansing and/or cosmetic treatment, and especially for the reinforcement, of the hair, more particularly of damaged and/or weakened hair, which comprises applying such a composition; this is optionally followed by a step of rinsing and/or optionally by a step of heat treatment.

The reason for this is that it is possible, after application of the composition according to the invention, to subject the keratin materials, especially the hair, to a heat treatment by heating at a temperature of preferably between 30 and 60° C. for 10 to 25 minutes. In practice this operation may be carried out using a styling hood, a hairdryer, an infrared radiation dispenser, or any other customary heating apparatus. It is possible to use, both as a means of heating and of smoothing of the hair, a heating iron at a temperature between 60 and 220° C., preferably between 120 and 200° C.

The invention is illustrated in greater detail in the examples below.

### Determination of the Molar Masses

The weight-average (Mw) and number-average (Mn) molar masses are determined by liquid chromatography by gel permeation or GPC (solvent THF, calibration curve established with linear polystyrene standards, refractometer detector). The dispersity index is calculated as follows: Ip=Mw/Mn

The GPC is carried out with Styragel HR4/7.8×300 mm columns sold by Waters WAT044225.

Detection is performed with a Waters 410 refractometer. The eluent is THF (tetrahydrofuran), at a flow rate of 1 ml/minute.

The volume injected is 50 microlitres, at 25° C.

Calibration is carried out using polystyrene standards.

### EXAMPLE 1

### Poly(L-lysine) Grafted with Polyethylene Glycol (PLL(20)-g-PEG(5) with g=3.5)

The procedure is that described by Spencer in J. Phys. Chem. B., Vol. 104, No. 14, 2000.

500 mg of poly(L-lysine) hydrobromide, with a molecular weight of 20,000 g/mol, are dissolved in 10 ml of 50 mM sodium borate buffer solution (pH 8.5).

1 g of methoxypoly(ethylene glycol) nitrophenyl carbonate with a molecular weight (Mw) of 5000 g/mol (sold by ShearwaterPolymers, Huntsville, Ala.) is dissolved with stirring in 2.5 ml of 50 mM sodium borate buffer solution (pH 8.5). This second solution is added to the first, polylysine solution.

Reaction is maintained at ambient temperature (25° C.) for 6 hours. The solution is subsequently dialysed (Spectra Por with a cutoff of 12,000 to 14,000 g/mol) for 24 hours against a phosphate-buffered saline solution PBS (pH 7.4; 0.1 ml of solution to 285 ml of buffer; 5% mOsm/kg H₂O) and then, for 48 hours, with deionized water.

This gives an aqueous solution of the polymer. The polymer is subsequently freeze-dried and stored at −20° C. under argon.

The product is characterized by ¹H NMR (D₂O, ppm) 1.35, 1.60, 1.68 (—CH₂—), 2.88 (—CH2—N—), 3.55 (PEG), 4.20 (—N—CHR—COO—).

The degree of grafting is determined by ¹H NMR, integrating the areas of the protons corresponding to the pendant chain of the lysine with those of the PEG. Furthermore, analysis by GPC chromatography is carried out with a refractive index detector: Shodex OHpak column, SB-804HQ, Alltech, Deerfield; Delory & King's carbonate-bicarbonate buffer, eluent: 0.2 M anhydrous sodium carbonate and 0.2 M sodium bicarbonate (pH 10).

The polymer obtained is a poly(lysine) whose backbone has a molecular weight of 20,000 g/mol, and the PEG grafts have a molecular weight (Mw) of 5 kDa (5000 g/mol). It is found that 1 lysine unit in 3.5 is grafted, corresponding to a degree of grafting of 28%.

Chain sequence within the polymer:

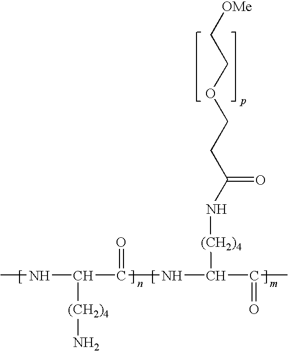

Structure of the polymer:

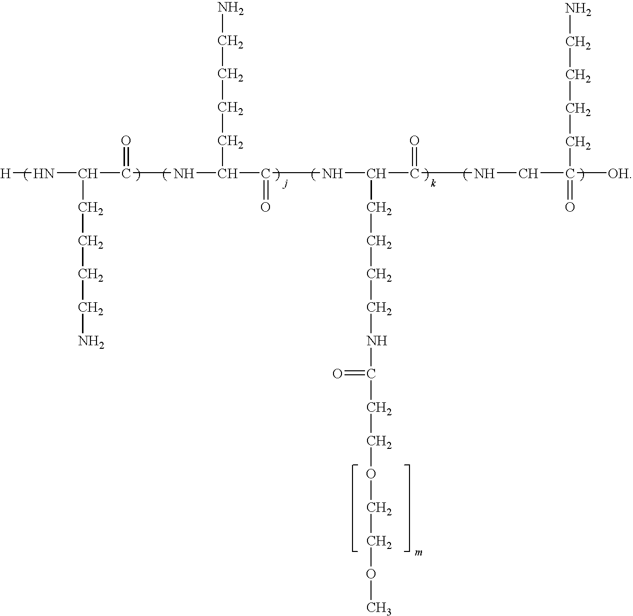

### EXAMPLE 2

### Poly(ornithine) with PEG Grafts

A solution is prepared containing 100 mg of poly(L-ornithine) in 60 ml of a 0.08 M sodium tetraborate buffer solution (pH 8.5). The solution is stirred for 3 hours and then admixed with—in 3 portions—200 mg of methoxypolyethylene glycol p-nitrophenyl carbonate (Mw 5000 g/mol). The mixture is stirred overnight with protection from light. The mixture is subsequently dialysed (5 litres of water) with 6 changes over a period of 24 hours.

This gives an aqueous solution of the desired polymer.

Chain sequence within the polymer obtained:

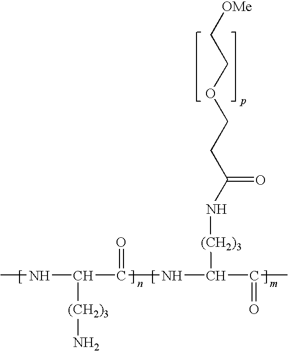

### EXAMPLE 3

### Evaluation

Locks of AS20 hair of 1 g which having undergone moderate bleaching (level of alkaline solubility: 20) are washed for 10 minutes with a 2% by weight solution of sodium lauryl sulphate. The wetted locks are treated over their entire length with 0.4 g of aqueous solution containing 5% by weight of active substance of polymer according to Example 1. They are kneaded for several minutes and then introduced into an oven at 60° C. for 30 minutes. The locks are subsequently rinsed with water for 10 seconds and then dried in the air.

A panel of testers evaluated the criteria of disentangling and of smoothing of the locks treated with the composition according to the invention, by comparison with untreated control locks.

The panel found that the treated locks exhibit a very good level of disentangling and of smoothing, and also of softness, on wet hair and also on dry hair after drying, relative to the untreated hair. These effects are still detectable after 5 shampooings.

It can be contemplated, without being bound by the present explanation, that the hair treatment according to the invention gives rise to modifications to the mass properties and surface properties of the damaged hair. The reason for this is that the damaged hair has a harsh surface texture which becomes smooth after treatment according to the invention; this effect remains for up to 5 consecutive shampooings after treatment.

### EXAMPLE 4

### Cosmetic Washing Composition

A shampoo is prepared which comprises (% by weight):

1% AS (active substance) of polymer of Example 1

12.5% of lauryl ether sulphate.

This gives a foaming solution which is applied to locks of AS 20 hair of 1 g, at a rate of 0.3 g of solution per lock. The solution is left on for 3 minutes and then rinsed off with water; the hair is disentangled and is found to be very easy to disentangle; it is dried under a hood at 40° C. for 15 minutes. The disentangling of the dry hair is found to be very good. The dried hair is very soft and shiny.

### EXAMPLE 5

### Styling Lotion

The following are introduced into a pump flask:

1% AS (active substance) of polymer of Example 1

6% of Styleze W 20 fixative polymer

2% of glycerol

qs preservative

qs to 100% water

This gives a styling lotion.

EXAMPLE 6

### Aerosol Styling Spray

An aqueous-alcoholic solution is prepared which comprises, in g of active substance:

| | |
|---|---|
| polyvinylpyrrolidone | 2.5 g |
| polymer of Example 2 | 0.65 g |
| glycerol | 10 g |
| ethanol | 15 g |
| water | 36 g |
| DME | 36 g |

### EXAMPLE 7

### Permanent Hairshaping Composition

A composition is prepared which comprises (% by weight):

| | | |
|---|---|---|
| thioglycolic acid | 9% | |
| 20% NH₃ aqueous ammonia | 9% | |
| ammonium carbonate | 4.5% | |
| cocoylamidopropylbetaine/glycerol mono- | 0.4% | AS |
| laurate (25/5) | | |
| EDTA | 0.4% | |
| polymer of Example 1 (AS) | 2% | |

| | |
|---|---|
| water | qs 100 g |

The above permanent reshaping compositions are applied for 15 minutes to wetted hair which beforehand has been rolled onto setting rollers, and then the compositions are rinsed off copiously with water. An 8-volume hydrogen peroxide solution with a pH of 3 is then applied for 5 minutes, after which the hair is rinsed again, freed of the rollers, and dried.

### EXAMPLE 8

### Conditioner

A composition is prepared which comprises (% by weight):

| | |
|---|---|
| behenyltrimethylammonium chloride at 80% | 1% |
| in a water/isopropanol mixture (15/85) | |
| cationic emulsion DC 929 (Dow Corning) | 4% |
| methyl p-hydroxybenzoate | 0.2% |
| polymer of Example 1 (AS) | 3% |
| water | qs 100% |

The conditioner is applied to wet hair. It is left on for 2 minutes and then rinsed off. It is very easy to remove and the disentangling of the hair is very good. The dried hair is very soft and shiny.

### EXAMPLE 9

### Treatment After Oxidizing Bleaching

An oxidizing bleaching operation is carried out using the commercial product Platifiz from L'Oréal on natural brown hair.

After the bleaching operation, the hair is rinsed with water before being treated for 5 minutes with a composition comprising a 2% aqueous solution of the polymer of Example 1 in the presence of a preservative. The treated hair is rinsed again thoroughly with water before being dried.

It is found that the hair treated with the composition of the invention is smoother, softer and easier to disentangle than the control hair (bleached and not treated).

### EXAMPLE 10

### Treatment After Hair Colouring

An oxidizing colouring operation is carried out using the commercial product Excellence Crème Châtain 4 from l'Oréal Paris on natural brown hair.

As an aftertreatment to the colouring, a composition is applied which comprises a 2% by weight aqueous solution of the polymer of Example 1 in the presence of a preservative, for 15 minutes, and then the hair is rinsed with water and dried.

### EXAMPLE 11

### Treatment After Straightening

A straightening composition is produced containing tetramethylguanidine at a concentration of 0.8 M in water, as an active straightening agent. The pH of the composition is 13.3. This composition is applied to naturally curly African hair for 15 minutes, at a temperature of 30° C. The hair is effectively straightened.

Then an aftertreatment composition is applied which comprises:

5% AS of the polymer of Example 1

5% AS of polyethylene glycol (1000 g/mol)

qs 100% water

The solution is applied for 15 minutes. The hair is subsequently rinsed and then dried.

It is found that the treated straightened hair is easy to comb and soft to the touch.

1. Hair treatment method for the hair, comprising the application of a cosmetic composition comprising, in a cosmetically acceptable medium, at least one polymer comprising a polymer backbone comprising amino acid monomer units with cationic function, and comprising hydrophilic grafts bonded covalently to said backbone, over all or part of said backbone, said hydrophilic grafts comprising the repetition of at least two hydrophilic monomer units of formula —CH₂—CH₂—O—.

2. Method according to claim 1, in which the cationic function is selected from:
guanidino and amidino moieties,
groups of formula —NRR′ or —N⁺RR′R″, where R, R′ and R″ represent, independently of one another, alternatively (i) a hydrogen atom, or (ii) an optionally aromatic, saturated or unsaturated, linear, branched or cyclic alkyl group containing 1 to 18 carbon atoms and able to contain 1 to 10 heteroatoms selected from O, N, S, F, Si and P, or (iii) R and R′ form, with the nitrogen atom, a first, optionally aromatic, saturated or unsaturated ring containing in total 5, 6, 7 or 8 atoms, and especially 4, 5 or 6 carbon atoms and/or 2 to 4 heteroatoms selected from O, S and N, it being possible for said first ring to be fused with one or more other, optionally aromatic, saturated or unsaturated rings each containing 5, 6 or 7 atoms, and especially 4, 5, 6 or 7 carbon atoms and/or 2 to 4 heteroatoms selected from O, S and N;
the groups: 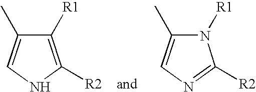
in which R1 and R2 may be, independently of one another, selected from hydrogen; an optionally aromatic, saturated or unsaturated, linear, branched or cyclic alkyl radical containing 1 to 18 carbon atoms and able to contain 1 to 10 heteroatoms selected from O, N, S, F, Si and P; or else it being possible for R1 and R2 together to form an optionally aromatic, saturated or unsaturated ring containing in total 5, 6, 7 or 8 atoms, especially 5 or 6 carbon atoms and/or 2 to 4 heteroatoms selected from O, S and N.

3. Method according to either of the preceding claims, in which the cationic function is selected from the groups NH₂, guanidino, and also

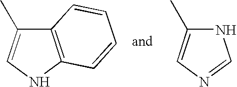

4. Method according to any of the preceding claims, in which the cationic amino acid monomers are selected from lysine, ornithine, arginine, histidine, glutamine and tryptophan, and a mixture thereof.

5. Method according to any of the preceding claims, in which the polymer backbone is selected from:
a homopolymer, more particularly of polylysine type, especially poly(E-lysine), poly(α-lysine), poly(L-lysine), poly(D-lysine), poly(D,L-lysine); or of polyornithine type, especially poly(L-ornithine), poly(D-ornithine), poly(D,L-ornithine); or of polyarginine type, especially poly(L-arginine), poly(D-arginine), poly(D,L-arginine); or of polyhistidine type, especially poly(L-histidine), poly(D-histidine), poly(D,L-histidine); or of polyglutamine type, especially poly(L-glutamine), poly(D-glutamine), poly(D,L-glutamine); or of polytryptophan type, especially poly(L-tryptophan), poly(D-tryptophan), poly(D,L-tryptophan);
a copolymer composed solely of cationic amino acid monomers, and especially of poly(lysine-co-ornithine) type;
a copolymer composed of cationic amino acid monomers in a mixture with additional monomers.

6. Method according to claim 5, in which the additional monomers are selected from:
non-cationic amino acid monomers, such as alanine, leucine or isoleucine, serine, threonine, cysteine, aspartic acid, glycine, valine, asparagine, phenylalanine, tyrosine, glutamic acid or proline;
polyalkylene glycol monomers, and especially polyethylene glycol;
vinyl monomers, especially (meth)acrylic and/or (meth)acrylamide monomers, and/or saccharide, ester, ether, carbonate, urethane and urea monomers, and a mixture thereof.

7. Method according to claim 6, in which the polymer backbone is selected from:
poly(lysine)-b-PEG block copolymers, poly(ornithine)-b-PEG block copolymers and poly(ornithine-co-serine)-b-PEG block copolymers;
polylysine-b-polyester and polylysine-b-polyurethane block copolymers;
poly(lactic acid-co-lysine) and poly(lysine-co-poly(N-hydroxypropylaminoethyl-D,L-aspartamide)) copolymers.

8. Method according to any of the preceding claims, in which the polymer backbone has a molecular weight (Mw) of between 1000 and 5,000,000, especially between 2000 and 1,000,000 and preferably between 3000 and 100,000 g/mol.

9. Method according to any of the preceding claims, in which the hydrophilic grafts are selected from polyalkylene oxides, and especially polyethylene oxide.

10. Method according to any of the preceding claims, in which each hydrophilic graft possesses a number-average molecular mass (Mn) of between 500 g/mol and 100,000 g/mol, preferably between 550 g/mol and 50,000 g/mol, more preferably between 1000 and 40,000 g/mol.

11. Method according to any of the preceding claims, in which the polymer has a degree of grafting of between 1% and 99%, especially between 2% and 50%, preferably between 3% and 45%, and more preferably still between 4% and 35%, and even between 5% and 25%.

12. Method according to any of the preceding claims, in which the polymer is present at from 0.01% to 30% by weight, preferably 0.1% to 20% by weight, especially 0.5% to 10% by weight, and even 1% to 5% by weight, relative to the weight of the cosmetic composition.

13. Method according to any of the preceding claims, further comprising a step of rinsing of the cosmetic composition and/or a step of heat treatment of the hair.

14. Method according to any of the preceding claims, comprising a method of making up, caring for, cleansing, colouring or shaping of the hair.
